(19) United States
(12) Patent Application Publication
(10) Pub. No.: US 2014/0273020 A1
(43) Pub. Date: Sep. 18, 2014

(54) SYSTEM FOR RAPIDLY DETECTING INFECTIOUS AGENTS USING A HYBRIDOMA-BASED BIOSENSOR

(71) Applicants: Thomas J. ZUPANCIC, Columbus, OH (US); J.D. Kittle, Columbus, OH (US); Lingchun Zeng, Columbus, OH (US); Srikanth Vedamoorthy, Columbus, OH (US); Min Mo, Columbus, OH (US)

(72) Inventors: Thomas J. ZUPANCIC, Columbus, OH (US); J.D. Kittle, Columbus, OH (US); Lingchun Zeng, Columbus, OH (US); Srikanth Vedamoorthy, Columbus, OH (US); Min Mo, Columbus, OH (US)

(73) Assignee: APPLIED BIOMOLECULAR TECHNOLOGIES, Columbus, OH (US)

(21) Appl. No.: 14/208,224

(22) Filed: Mar. 13, 2014

Related U.S. Application Data
(60) Provisional application No. 61/779,362, filed on Mar. 13, 2013.
(60) Provisional application No. 61/780,138, filed on Mar. 13, 2013.

(51) Int. Cl. G01N 33/569

(52) U.S. Cl. CPC G01N 33/56916
USPC 435/7.37; 435/340; 435/287.2

(57) ABSTRACT

A system for detecting infectious agents in biological samples in real time that includes a sample to be tested for at least one specific infectious agent; and at least one biosensor, wherein the biosensor is operative to detect a specific infectious agent in the sample to be tested; wherein the biosensor emits a detectable signal when it reacts with the specific infectious agent; wherein the biosensor is a hybridoma cell that naturally expresses an endogenous anti-target antigen specific IgM; and wherein the hybridoma cell has been converted to a B cell receptor biosensor by introducing a detectable reporter gene into the cell.

### CROSS-REFERENCE TO RELATED APPLICATIONS

This patent application claims the benefit of U.S. Provisional Patent Application Ser. No. 61/779,362 filed on Mar. 13, 2013 and entitled “Rapid Detection System for Infectious Agents” and Ser. No. 61/780,138 filed on Mar. 13, 2013 and entitled “Biosensor-Based Rapid Detection System for Infectious Agents, the disclosures of which are hereby incorporated by reference herein in their entirety and made part of the present U.S. utility patent application for all purposes.

### BACKGROUND OF THE INVENTION

The described invention relates in general to a system for detecting contaminants in biological samples and more specifically to a system for detecting infectious agents or pathogens in food samples in real time that includes a sample to be tested for a specific infectious agent; and a biosensor, wherein the biosensor is operative to detect at least one specific infectious agent in the sample to be tested; and wherein the biosensor emits a detectable signal when it reacts with the specific infectious agent.

In generic terms, a biosensor is a system or device for the detection of an analyte that combines a sensitive biological component with a physicochemical detector component. The components of a typical biosensor system include a biological element, a transducer or detector element, and associated electronics or signal processors that display test results in a meaningful and useful manner. The biological element includes biological material such as tissue, microorganisms, organelles, cell receptors, enzymes, antibodies, nucleic acids, and the like that may be created by known biological engineering processes. The transducer or detector element works in a physicochemical manner (e.g., optical, piezoelectric, and/or electrochemical) that transforms the signal resulting from the interaction of the analyte with the biological element into another signal that can be more easily measured and quantified. Biosensors originated from the integration of molecular biology and information technology (e.g., microcircuits, optical fibers, etc.) to qualify or quantify biomolecule-analyte interactions such as antibody-antigen interactions. Considering that there is great demand for rapid, sensitive, easy-to-handle, and cost-effective detection tools to detect infectious agents, pathogens or/and toxins in food (see, for example, Mead et al., Food Related Illness and Death in the United States, Emerging Infectious Diseases; Vol. 5, No. 5, September-October 1999 (607-625), which is incorporated by reference herein, in its entirety), there is an ongoing need for the utilization of biosensors in real-time, field-portable devices and instruments for the detection and identification of infectious agents, pathogenic microorganisms, toxins, and other contaminants in foods.

### SUMMARY OF THE INVENTION

The following provides a summary of certain exemplary embodiments of the present invention. This summary is not an extensive overview and is not intended to identify key or critical aspects or elements of the present invention or to delineate its scope.

In accordance with one aspect of the present invention, a biosensor is provided. This biosensor includes a hybridoma cell that naturally expresses an endogenous anti-target antigen specific IgM; wherein the hybridoma cell has been converted to a B cell receptor biosensor by introducing a detectable reporter gene into the cell; wherein the biosensor is operative to detect a specific infectious agent in a sample to be tested; and wherein the biosensor emits a detectable signal when it reacts with the specific infectious agent.

In accordance with another aspect of the present invention, a first system for rapidly detecting infectious agents in biological samples is provided. This system includes at least one sample to be tested for at least one specific infectious agent; and at least one biosensor. The biosensor is operative to detect a specific infectious agent in the sample to be tested and emits a detectable signal when it reacts with the specific infectious agent. The biosensor is also a hybridoma cell that naturally expresses an endogenous anti-target antigen specific IgM and that has been converted to a B cell receptor biosensor by introducing a detectable reporter gene into the cell.

In accordance with still another aspect of the present invention, a second system for rapidly detecting one or more infectious agents in biological samples is provided. This system includes at least one sample to be tested for at least one specific infectious agent; and at least one biosensor. The biosensor is operative to detect a specific infectious agent in the sample to be tested and emits a detectable signal when it reacts with the specific infectious agent. The biosensor is also a hybridoma cell that naturally expresses an endogenous anti-target antigen specific IgM and that has been converted to a B cell receptor biosensor by introducing a detectable reporter gene into the cell, wherein the detectable reporter gene is an aequorin reporter gene.

Additional features and aspects of the present invention will become apparent to those of ordinary skill in the art upon reading and understanding the following detailed description of the exemplary embodiments. As will be appreciated by the skilled artisan, further embodiments of the invention are possible without departing from the scope and spirit of the invention. Accordingly, the drawings and associated descriptions are to be regarded as illustrative and not restrictive in nature.

### BRIEF DESCRIPTION OF THE DRAWINGS

The accompanying drawings, which are incorporated into and form a part of the specification, schematically illustrate one or more exemplary embodiments of the invention and, together with the general description given above and detailed description given below, serve to explain the principles of the invention, and wherein:

FIG. 1 is a chart illustrating the effectiveness of the system of the present invention with regard to detecting the presence of one or more infectious agents in a biological sample. The Y-axis represents the amount of light flash, and the X-axis represents the time in seconds;

FIG. 2 is a graph which is a graph illustrating the selection of high flash capable cells, shows the concentration of antibiotics used for selection and the light emitted (photon counts per second);

FIG. 3 is a graph showing that modified 1E7 cells demonstrate high specificity to O111 bacteria and O111 derived LPS;

FIG. 4 is a graph is a graph showing the ability of the biosensor cells of this invention to detect O111 lipopolysaccharide; and

FIG. 5 is a flow chart of a system and process for testing a biological sample for the presence of one or more infectious agents, in accordance with an exemplary embodiment of the present invention.

### DETAILED DESCRIPTION OF THE INVENTION

Exemplary embodiments of the present invention are now described with reference to the Figures. Reference numerals are used in the detailed description to refer to the various elements. Although the following detailed description contains many specifics for the purposes of illustration, a person of ordinary skill in the art will appreciate that many variations and alterations to the following details are within the scope of the invention. Accordingly, the following embodiments of the invention are set forth without any loss of generality to, and without imposing limitations upon, the claimed invention.

As previously discussed, the present invention provides a system for accurately and rapidly (i.e., typically within one to five minutes) detecting infectious agents in biological and non-biological samples, particularly samples derived from beef, poultry, fish, or vegetable matter, although other biological materials may be analyzed using this invention. The biosensor is operative to detect a specific infectious agent in the sample to be tested by emitting a detectable signal when it reacts with the infectious agent. This system provides very high sensitivity without the need to culture infectious agents such as bacteria obtained from samples prior to testing. In some embodiments, the system of the present invention is capable of detecting a predetermined number of cells of an infectious agent such as, for example, one thousand cells or less. Some embodiments of this invention are expected to be able to detect very small numbers of cells, down to even a single cell of a particular infectious agent. In an exemplary embodiment, the specific infectious agent is Escherichia coli, although other infectious agents (such as Salmonella, Listeria, and Campylobacter), toxins, and various contaminants may be detected with the present invention. Escherichia coli O157:H7, O26, O45, O103, O111, O121, and O145, in either separate assays or multiplexed assays, may all potentially be detected using this invention. This invention provides a biosensor that is based on a design that simplifies the construction process and improves the performance characteristics of the biosensor cell over prior art systems.

In accordance with this invention, an exemplary biosensor includes a human B lymphocyte engineered to express a bioluminescent protein and at least one membrane-bound antibody specific for a predetermined infectious agent. With regard to this type of biosensor, cell-based biosensor (CBB) systems that incorporate whole cells or cellular components respond in a manner that can offer insight into the physiological effect of an analyte. As will be appreciated by those skilled in the art, cell-based assays (CBA) are emerging as dependable and promising approaches for detecting the presence of pathogens in clinical, environmental, or food samples because living cells are known to be extremely sensitive to modulations or disturbances in “normal” physiological microenvironments. Therefore, CBBs have been employed to screen and monitor “external” or environmental agents capable of causing perturbations of living cells (see, for example, Banerjee et al., Mammalian cell-based sensor system, Adv. Biochem. Eng. Biotechnology, 117:21-55 (2010), which is incorporated herein by reference, in its entirety). Compared with traditional detection methods (e.g., immunoassays and molecular assays such as PCR), a biosensor provides distinct advantages such as: (i) speed, i.e., several seconds to less than 10 minutes; (ii) increased functionality, which is extremely important for reporting active components such as live pathogens or active toxins; and (iii) ease of scale-up for performing high-throughput screening.

A critical component of the adaptive immune system is the B lymphocyte, which is a type of white blood cell that begins its development in the bone marrow and as such is involved in “humoral” immunity. Upon encountering an antigen, a B lymphocyte differentiates into a plasma cell, which then secretes immunoglobulin that functions as an antibody to the antigen. B cells are distinguishable from other lymphocytes, such as T cells and natural killer cells (NK cells) by the presence of a protein on the outer surface of the B cell known as a B cell receptor (BCR). This specialized receptor protein allows a B cell to bind to a specific antigen; thus, the principal functions of B cells are to (i) make antibodies against antigens; (ii) perform the role of antigen-presenting cells (APCs); and (iii) develop into memory B cells after activation by antigen interaction. Given these characteristics, biosensor or pathogen sensor systems may utilize or incorporate B lymphocytes for the purpose of detecting specific antigens. For example, B cells lines have been engineered to express cytosolic aequorin, a calcium-sensitive bioluminescent protein from the Aequoria victoria jellyfish, in addition to membrane-bound antibodies specific for certain pathogens. In this system, cross-linking of the antibodies by even low levels of the appropriate pathogen elevated intercellular calcium concentrations within seconds, causing the aequorin to emit a detectable signal of light.

An aequorin-based biosensor system is utilized with certain embodiments of the present invention. Aequorin is a 21-kDa calcium-binding photoprotein isolated from the luminous jellyfish Aequorea victoria. Aequorin is linked covalently to a hydrophobic prosthetic group (coelenterazine) and upon calcium (Ca2+) binding, aequorin undergoes an irreversible reaction, and emits blue light (at 469 nm). The fractional rate of aequorin consumption is proportional, in the physiological pCa range, to [Ca2+]. Application of the aequorin-Ca2+ indicator to detect E. coli contamination in food products was reported in 2003 (see, Rider et al. A B cell-based sensor for rapid identification of pathogens, Science, 301(5630):213-5 (2003), which is incorporated by reference herein, in its entirety). Engineered B lymphocytes were used to express antibodies that recognize specific bacteria and viruses and aequorin, which emits light in response to the calcium flux triggered by the binding of a cognate target to the surface-antibody receptor. The resulting biosensor cell emitted light within minutes in the presence of the targeted microbes. To create these biosensor cells, antibody heavy and light chains with variable regions were cloned and expressed in a B-lymphocyte cell line. The resulting immunoglobulins become part of a surface B-cell-receptor complex, which includes the accessory molecules immunoglobulina (Igα, or CD79a) and immunoglobulinβ (Igβ, or CD79b). When the complex is cross-linked and clustered by polyvalent antigens, such as microbes, a set of signaling events quickly leads to changes in the intracellular calcium-ion concentration, which then causes aequorin to emit light. This mechanism essentially hijacks the B-cell's intrinsic capacity to specifically recognize the antigen presented in the E. coli by the B-cell membrane IgG antibody, and this binding triggers a transient Ca2+ influx to cytosol, which binds the aequorin proteins engineered in this B-cell, and subsequently emit blue light (see, Relman, Shedding light on microbial detection, N England J Med, 349(22):2162-3 (2003), which is incorporated by reference herein, in its entirety).

Selection of an appropriate B cell is an important aspect of this invention and any proposed cell line should be tested to confirm that the B cell receptor signaling pathway is fully functional. Individual B cell clones having the aequorin gene should be tested to identify a particular clone with high aequorin activity as significant variation from one clone to the next is possible (see, generally, Calpe et al., ZAP-70 enhances migration of malignant B lymphocytes toward CCL21 by inducing CCR7 expression via IgM-ERK1/2 activation, Blood, 118(16):4401-10 (2011) and Cragg et al., Analysis of the interaction of monoclonal antibodies with surface IgM on neoplastic B-cells, Br J Cancer, 79(5/6): 850-857 (1999), both of which are incorporated by reference herein in their entirety). A high-aequorin expressing B cell is important for achieving high levels of sensitivity when using this detection system. In an exemplary embodiment, the receptor response for the biosensor was verified by using the Ramos human B cell line. Ramos cells were first transfected with the aequorin gene and the transfected cells were then selected for aequorin expression for two weeks. After that, mixed Ramos cells were charged with coelenterazine (CTZ), stimulated with anti-IgM Ab, and flash signal elicited by the reaction was captured by a luminometer. As shown in FIG. 1, anti-IgM stimulation causes an expected sizeable and prolonged flash (from 45 to 65 seconds). In FIG. 1, the Y-axis represents the amount of light flashing, and the X-axis represents the reaction time in seconds. At 30 seconds, the anti-IgM solution was injected into the Ramos-Aeq cell solution; the first spike (from 30-37 seconds) is noise signal, and the second larger and longer peak is the biological response to anti-IgM stimulation. To improve the overall signal/noise ratio, the CTZ was removed from the CTZ-charged Ramos-Aeq cells. Removal of CTZ from the cell solution decreases noise signal from around 150 to about 50 without significantly compromising the amount of the true peak signal.

In accordance with this invention, an exemplary protocol for cell handling and flash-testing includes: (i) culturing Ramos-Aeq cells with regular culture medium and keeping these cells healthy (i.e., viability >98%); (ii) charging the Ramos-Aeq cells with CTZ at a final concentration of 2 μM, the cell density being 1-2 million per milliliter; (iii) charging the cells at 37° C. with 5% CO₂ in an incubator for at least 3 hours; (iv) removing the charging medium containing CTZ; (v) flash testing by taking 200 μl cell solution plus 30 μl stimulants (anti-IgM) and reading with a luminometer; and (vi) confirming CTZ and aequorin functionality by adding 30-40 uL Digitonin (770 μM).

With regard to the biosensor component, an exemplary embodiment of the present invention utilizes a hybridoma cell based biosensor. Hybridomas are immortalized cells derived from the fusion of a specific antibody-producing B cell with a myeloma (B cell cancer) fusion partner that is selected for its ability to grow in tissue culture and for an absence of antibody chain synthesis. The antibodies produced by the hybridoma are all of a single specificity and are therefore monoclonal antibodies (in contrast to polyclonal antibodies). As previously discussed, mammalian B cell based biosensors have previously been constructed by introducing into B cells genes encoding the heavy and light chains for a membrane bound form of IgM antibody as well as a gene encoding a detectable reporter protein. Because B cells already express the alpha and beta chains of the B cell receptor (BCR), the modified B cells that are created this way can bind to the antigen recognized by the introduced antibody and trigger the B cell receptor signal transduction pathway. Activation of this pathway leads to a transient increase in intracellular calcium concentration; thus, it is possible to generate a detectable signal in response to binding to a target antigen by using a reporter protein that is activated when the calcium concentration is increased.

The present invention provides a mammalian cell biosensor that is useful for the detection of infectious pathogens or agents in environmental samples and that is based on a design that simplifies the construction process and improves the performance characteristics of the biosensor cell over prior art systems. Rather than introducing a target antigen specific IgM antibody into a B cell expressing the B cell receptor, the method of the present invention begins by isolating a hybridoma cell that is selected because it naturally expresses an endogenous anti-target antigen specific IgM. This cell is then converted to a BCR biosensor in a single step by adding a gene encoding a detectable reporter gene.

The method of this invention eliminates a number of problems that are inherent in the traditional biosensor design. First, the addition of multiple genes into a host cell requires the use of antibiotic resistance genes and corresponding antibiotics that are added to the culture medium in order to select for and maintain the introduced genes in the cell. If independent gene delivery vehicles are used for reporter, antibody heavy chain and antibody light chain, three different antibiotics are required, thereby adding to the cost of production and increasing the undesirable use of large amounts of antibiotics in the cell production process. Advantageously, the hybridoma approach of this invention only requires a single gene delivery vehicle/selectable marker gene/antibiotic. Additionally, B cells are relatively difficult to engineer using contemporary genetic engineering techniques. More specifically, B cells are relatively refractory to standard gene delivery techniques such as chemical transformation and electroporation. This characteristic of B cell lines complicates the task of cell engineering. Most significantly, many promoter elements required for efficient transgene expression perform poorly in B cells making it necessary to test alternative expression constructs for the engineered antibody heavy and light chains. This complicates the need to achieve an appropriate balance between heavy and light chain expression. In contrast, the hybridoma cells of the present invention express the target antigen specific using the endogenous heavy and light gene promoters and naturally achieve appropriate relative amounts of heavy and light chain protein, overcoming this problem. Finally, the hybridoma cell approach eliminates problems with cell line stability that typically exist with genetically modified cell lines. Cells that have been engineered using traditional methods are prone to deletion events over multiple generations that ultimately result in the loss of the key transgenes that are required for functionality. Furthermore, transgenes that are introduced into a host cell using traditional methods are susceptible over time and through multiple cell divisions to gene silencing, such as by DNA methylation. Since the hybridoma cells of the present invention use the endogenous antibody genes, these problems are presumably eliminated.

In accordance with an exemplary embodiment of the present invention, a hybridoma cell based biosensor for pathogenic E. coli strain O111 was constructed using the following approach. An anti-O111 E. coli IgM expressing hybridoma cell was identified by inoculating mice with the insoluble membrane containing fraction from a preparation of O111 E. coli cells. These mice were further boosted with purified lipopolysaccharide (LPS) that had been isolated from the outer membrane of O111 E. coli cells. Once the mice had generated a suitable anti-O111 specific antibody titre, B cells were isolated from the mice and fused to a standard myeloma fusion partner following standard protocols. Individual clones were then generated from this population of hybridomas. These clones were screened to identify a particular clone that expressed and anti-O111 E. coli IgM antibody (clone 1E7), which was used to construct a functional E. coli O111 specific biosensor. In addition, a second hybridoma cell line that also expresses an alternative anti-E. coli O111 IgM antibody was isolated (8C6) and this cell line was also was used to construct a second E. coli O111 biosensor cells. A third hybridoma cell line that expressed a different anti-E. coli O111 IgM (7G12) was also isolated, as well as a hybridoma cell line that expresses and anti-E. coli 0157 IgM (1C1).

Specificity of the generated hybridoma was confirmed by ELISA. LPS (500 ng/ml) of O157, O127, O111, O26, Klebsiella, Salmonella bacteria's were coated on ELISA plate. Culture media from O111 hybridomas (1E7, 7G12) cells was applied to each well and incubated for 1 hour at room temperature. After several washings, anti-mouse IgM-HRP conjugate was added. TMB reagent (substrate) was added and the color developed was read at 450 nm, after adding 1N HCl. Table 1, below, shows the high specificity of 1E7, 7G12 hybridoma to O111 LPS and the absence of cross-reactivity with other bacterial LPS.

TABLE 1
High specificity of 1E7, 7G12 hybridoma to O111 LPS.

| LPS | O157 | O127 | O111 | O26 | Klebsiella | Salmonella |
|---|---|---|---|---|---|---|
| 1E7 | − | − | + | − | − | − |

With regard to immunoglobulin isotype specificity of 1E7, immunoglobulin isotype of 1E7 hybridoma was analyzed by ELISA, using ‘μ’ (and ‘γ’) chain specific antibodies. Validity of ‘γ’ chain response to O111 LPS was cross checked with Salmonella LPS. High reactivity of ‘μ’ chain antibody to 1E7 cell culture media confirmed the specific immunoglobulin M (IgM) expression (see TABLE 2, below).

TABLE 2
Immunoglobulin isotype specificity of 1E7.

| Clone name | LPS of E.coli O111 | | LPS of Salmonella |
|---|---|---|---|
| 1E7 | γ chain | μ chain | γ chain |
| | 0.471 | 3.000 | 0.555 |

Clone 1E7 was analyzed by RT PCR to demonstrate that this cell line expressed the membrane-bound form of IgM as well and the alpha and beta chains of the B cell receptor. Then the expression of the IgM antibody heavy and light chains on the outer membrane of the hybridoma cells was visualized by immunohotochemistry using a fluorescently labeled anti-murine IgM antibody. Finally, the presence of IgM antibodies on the surface of the hybridoma cell was confirmed by Flourescence-activated Cell sorting. A plasmid expression vector containing an aequorin reporter gene (on pcDNA3.1 with G418 selectable marker) was then introduced into the cells by electroporation. The cells were then subcultured in the presence of G418 antibiotic. The G418 resistant cells obtained were tested for expression of the luminescent aequorin reporter gene by charging the cells with coelentarizine followed by addition of digitonin using standard methods. The cells were then analyzed in a luminometer to demonstrate a rapid flash, indicating that active aequorin enzyme was present. Then, IgM mediated B cell receptor signaling was demonstrated by charging the cells with coelentarizine as above, followed by addition of and anti-murine IgM antibody. When analyzed in a luminometer, these cells showed an IgM-BCR mediated flash, demonstrating the key function of the biosensor cell.

It has been observed that plasmid expression vectors such as pcDNA3.1 that use the CMV promoter to drive expression of the aequorin transgene in B cell and hybridoma cells do not function as well in these cell types as they do in standard mammalian host cells such as CHO cells and HEK293 cells. To address this limitation, an alternative hybridoma cell biosensor for E. coli O111 was also constructed by inserting the aequorin gene adjacent to the translation factor EF1 alpha promoter in the pVITRO plasmid expression vector and then inserting this vector into an alternative anti-O111 IgM expressing hybridoma cell line. The resulting transfected cells expressed approximately 10 times as much aequorin as the same cells transfected with the aequorin gene expressed from the CMV promoter in pcDNA3.1, demonstrating that the appropriate choice of promoter element is key to construction of a sensitive biosensor cell. In particular, it is desirable to construct expression vectors using promoter elements that are specifically and highly expressed in B cell derived cell lines such as hybridoma cells. Thus an immumoglobulin heavy chain promoter is a useful genetic element for constructing a sensitive hybridoma biosensor. The activity of such a promoter can be further increased by incorporating an enhancer element such as the E mu enhancer from the antibody gene variable region. Even greater promoter activity can be achieved by also incorporating functional elements from the antibody gene 3′ regulatory region. The key is to drive expression of the aequorin reporter protein using a promoter that is highly active in hybridoma cells.

It is also useful to increase the expression of the B cell receptor alpha and beta proteins in the hybridoma cells since it has been shown that coexpression of these proteins is required for the transport of function IgM antibodies to the plasma membrane. This can be accomplished by constructing a murine B cell receptor alpha-beta fusion protein where a virus derive 2A peptide sequence with a furin cleavage site at the C terminus of the first B cell receptor protein (the N terminus of the 2A peptide). This fusion protein can then be inserted into a dual promoter vector together with the aequorin reporter gene and then transfected into the hybridoma cells, allowing for insertion of all the key transgenes using a single gene delivery vehicle.

To construct an E. coli O111 specific biosensor 1E7 hybridoma cells were manipulated as follows. High expression of aequorin (photon emitter, which reflects the sensor's response) in hybridoma cells was achieved by selecting the aequorin gene electroporated cells. 3.0 μg of aequorin expression plasmid (pcDNA 3.1(+)-Aeq) was electroporated to 1E7 hybridoma cells (1×10⁶). After 3 days of transfection, varying concentration of neomycin antibiotic (1.2 to 6 mg/mL) was added and selected for 60 days. Selected hybridoma cells were charged with coelenterazine (CTZ) for 4 hrs and the emission of photons quantified, after the addition of digitonin. FIG. 2, which is a graph illustrating the selection of high flash capable cells, shows the concentration of antibiotics used for selection and the light emitted (photon counts per second).

The ability of the 1E7+ aequorin biosensor cells to specifically detect E. coli O111 cells and lipopolysaccharide was then demonstrated and FIG. 3 is a graph showing that these modified 1E7 cells show high specificity to O111 bacteria and O111 derived LPS. 1E7 cells which express high mIgM and aequorin were charged with (2 μM) CTZ for 4 hours and challenged with different bacterial strains and LPS. Different strains (O111, O26, O157 and DH5α) of bacteria (3×10̂8) were used to check the specificity of 1E7 cells. Results demonstrated that the 1E7 hybridoma derived biosensor cells could specifically detect O111 E. coli cells. FIG. 4 is a graph showing the ability of the biosensor cells of this invention to detect O111 lipopolysaccharide (LPS was then demonstrated as follows). LPS was released and quantified from O111, O157, O26, O127, Klebsiella, Salmonella as per the ToxinSensor Chromatogenic LAL Endotoxin Assay Kit protocol. 1 mg/mL LPS was used to perform the E. coli detection experiment. Goat anti-mouse IgM antibody was used as positive control. The results showed that the 1E7 hybridoma cells that expressed the aequorin reporter protein could specifally detect the O111 antigen. Thus, the utility as E. coli serotype specific biosensors of IgM expressing hybrodoma cells that have been modified to express an aequorin reporter gene has been demonstrated.

As previously indicated, in some embodiments, the bioassay described herein may be carried out in a testing subunit or test cartridge designed for use with a bench-top or portable testing system and device such as that disclosed in U.S. patent application Ser. No. 13/711,296, which is incorporated by reference herein, in its entirety. The test cartridge, which may be a single-use, disposable item, receives both the sample and the biosensor, and introducing the biosensor into the test cartridge mixes the sample and the biosensor in a predictable manner. The test cartridge further includes a reaction chamber for receiving the sample and the biosensor, wherein the reaction chamber has a predetermined internal geometry and has been further adapted to minimize or eliminate background noise for the purpose of improving the overall signal to noise ratio. A least one stabilizer may located in the reaction chamber for minimize shear force damage to the sample and biosensor during the mixing process.

In an exemplary embodiment (not shown in the Figures), the reaction chamber and fluid channels that lead to the reaction chamber within the test cartridge are designed to achieve several objectives. An inlet channel for fluid entering the reaction chamber is a tubular shape and the diameter of the tube is relatively small and tapers to become smaller at the inlet to the reaction chamber. This increases the velocity of fluid entering the reaction chamber and promotes more vigorous and homogenous mixing due to the bulk motion of the reagents within the reaction chamber. It is desirable to mix the reagents and sample in a way to promote mixing beyond molecular diffusion, in order to minimize the duration of the test by ensuring that any infectious agent present in the sample rapidly encounters the biosensor. The inlet channel may be offset from the central axis of the reaction chamber to promote a clockwise or counterclockwise rotational motion of the reagents around the central axis of the test chamber as the fluids are mixed in order to increase homogeneity of the mixture. The inlet channel is also approximately tangent to the interior surface of the reaction chamber for allowing incoming fluid to travel from the inlet channel to the reaction chamber while remaining in contact with the side surface of the reaction chamber, which allows for a minimally turbulent flow and minimal introduction of air bubbles into the mixed fluids. Bubbles are undesirable due to the unpredictable refraction of light they cause as light emitted by the reagents travels through bubbles within the mixed reagents or on the surface of the mixed reagents. The axis of the inlet channel may be angled above horizontal (e.g., about 30 degrees) to provide a partially downward direction to the incoming fluid flow to ensure that the reagent is mixed with the fluid residing at the bottom of the reaction chamber. Alternatively, the reagents may be introduced to the test chamber using alternative fluid delivery means such as a vertical channel to deliver the reagents to the bottom of the reaction chamber, or delivering the fluid directly on the central axis of the test chamber in order to create a column of reagent flowing into the test chamber thereby promoting mixing through entrainment.

The shape (i.e., predetermined geometry) of the reaction chamber may be a revolved section facilitating clockwise or counterclockwise motion of the mixing fluids around the central axis of the reaction chamber. Alternatively, if desired, a reaction chamber shape other than a revolved section such as a rectangular or irregular shape may be utilized. In one embodiment, the revolved section used to form the reaction chamber is a portion of an ellipse for facilitating the collection of stray light emitted by the reagents and reflecting this light toward the surface of the detector, which may be a photomultiplier tube (PMT) (Hamamatsu). The surface of the reaction chamber may be reflective, in order to enhance the light collection properties of the elliptical shape. In some embodiments, the maximum diameter of the surface of the PMT is limited to achieve a maximum signal to noise ratio of the output of the system. The diameter of the reaction chamber may be designed to approximately match the diameter of the PMT, which influences the elliptical shape that can be achieved in a reaction chamber designed to hold a specific volume of fluids. Due to the constrained elliptical shape, the reaction chamber surface color may be a partially diffusing white due to the additional light collection that occurs when light that would not otherwise be reflected directly to the PMT surface is partially diffused by the white surface and a fraction of this is directed toward the PMT surface. Alternatively, other surface finishes and materials such as a near-mirror finish aluminum, or a transparent material could be used if desired. Further, it is desirable for the reaction chamber material to be minimally phosphorescent, in order to prevent light emitted from the reaction chamber itself from eclipsing any emitted light from the reagents and preventing detection. Although white polymeric materials such as acrylonitrile butadiene styrene or other such polymeric materials have been found to exhibit a low level of phosphorescence, the additional light collection provided by the combination of light reflection and diffusion has been found to be a benefit to the signal to noise ratio of the light sensing circuit output.

In some embodiments of the invention, the stabilizer included in the test chamber is Pluronic F68, which is used in cell culture as a stabilizer of cell membranes by protecting from membrane shearing and additionally as an anti-foaming agent. Certain embodiments of this invention also include at least one additive located in the test chamber for minimizing the formation of bubbles in the test chamber during mixing of the sample and the biosensor. This additive may further include a surfactant. Some embodiments also include a device for disrupting individual cells of the infectious agent prior to mixing the sample with the biosensor for purposes of amplifying the light signal generated by the biosensor reacting with the infectious agent. An example of such a device would be a sonicator. The detector for detecting and amplifying the signal generated by the biosensor may be a photomultiplier tube (Hamamatsu) having an active surface, and wherein the size (i.e., diameter) of the active surface has been optimized to reduce background noise and increase the signal to noise ratio of the detected signal. The system of the preset invention may also include at least one data processing component in communication with the testing unit for receiving information therefrom. The at least one data processing component further includes both computer hardware and software for organizing, interpreting, and storing the information received from the testing unit and for presenting the information in a useful and meaningful manner.

FIG. 5 provides a flow chart of a system and process for testing a biological sample for the presence of one or more infectious agents, in accordance with an exemplary embodiment of the present invention, including: (i) growing appropriate engineered B cells at process step 10; (ii) charging the cells with coelenterazine at process step 12; (iii) removing excess coelenterazine at process step 14; (iv) adding a cell stabilizer (e.g. pluronic F68) at process step 16; (v) adding a cryopreservative (e.g., dimethyl sulfoxide (DMSO) at process step 18; (vi) loading biosensor cells into a disposable test cartridge at process step 20; (vii) loading positive control samples into the disposable cartridge (anti-IgM, digitonin, etc.) at process step 22; (viii) freezing and storing the frozen cartridge (below −40° C.) at process step 24; (ix) thawing the cartridge at process step 26; (x) inserting the cartridge into a detection device at process step 28; (xi) preparing a test sample having an infectious agent therein at process step 30 by fragmenting the infectious agent (using sonication, pressure gradient, and/or enzyme treatment, for example); (xii) injecting the sample into the reaction chamber at process step 32; (xiii) activating the detection device analyze sample at process step 34; and (xiv) performing a positive control at process step 36.

As previously discussed, proper operation of the system of the present invention includes reducing background noise to a minimum. This may be accomplished by (i) removing CTZ after charging the non-B cell (CTZ spontaneously oxidizes and creates background photons); (ii) adding EDTA (or other chelating agent) to chelate extracellular calcium; (iii) shielding the PMT from electromagnetic radiation by, for example, positioning the PMT at an appropriate distance from sources of background EM radiation; (iv) using a plastic material with minimal fluorescence (plastic glows in the dark and the appropriate selection/coating of the plastic reduces this noise; and (v) using a chamber surface coating the blocks photons from the plastic and that does not emit photons itself. Other important aspects include: (i) adjusting the cell concentration to an optimal level to balance the need for many cells to detect rare events with the fact that cells themselves absorb light and interfere with transmission of the signal to the detector; and (ii) incorporating an automatic positive control into the test process using, for example, an anti-IgM antibody or a generic calcium influx inducer such as digitonin. In a two-step process, step one includes mixing the biosensor cells with the sample to detect infectious agents, and step two includes adding the positive control inducer to validate that the biosensor cells well fully active. Further biosensor signal amplification may be achieved by fragmenting the individual cells of the infectious agent, using for example: (i) an enzyme such as lipase to release O antigens from the cell surface (part of LPS); (ii) sonication to fragment the cells; (iii) a French Press or equivalent to fragment the cells; or (iv) a chemical treatment to release LPS from the cells.

While the present invention has been illustrated by the description of exemplary embodiments thereof, and while the embodiments have been described in certain detail, it is not the intention of the Applicant to restrict or in any way limit the scope of the appended claims to such detail. Additional advantages and modifications will readily appear to those skilled in the art. Therefore, the invention in its broader aspects is not limited to any of the specific details, representative devices and methods, and/or illustrative examples shown and described. Accordingly, departures may be made from such details without departing from the spirit or scope of the applicant's general inventive concept.

What is claimed:

1) A biosensor, comprising: a hybridoma cell that naturally expresses an endogenous anti-target antigen specific IgM; wherein the hybridoma cell has been converted to a B cell receptor biosensor by introducing a detectable reporter gene into the cell; wherein the biosensor is operative to detect a specific infectious agent in a sample to be tested; and wherein the biosensor emits a detectable signal when it reacts with the specific infectious agent.

2) The biosensor of claim 1, wherein the detectable reporter gene is an aequorin reporter gene.

3) The biosensor of claim 1, wherein the specific infectious agent is Escherichia coli.

4) The biosensor of claim 1, wherein the sample to be tested is derived from beef, poultry, fish, or vegetable matter.

5) The biosensor of claim 1, wherein the detectable signal further includes light.

6) A system for rapidly detecting infectious agents in biological samples, comprising:
(a) at least one sample to be tested for at least one specific infectious agent; and
(b) at least one biosensor, wherein the biosensor is operative to detect a specific infectious agent in the sample to be tested; wherein the biosensor emits a detectable signal when it reacts with the specific infectious agent; wherein the biosensor is a hybridoma cell that naturally expresses an endogenous anti-target antigen specific IgM; and wherein the hybridoma cell has been converted to a B cell receptor biosensor by introducing a detectable reporter gene into the cell.

7) The system of claim 6, further comprising:
(a) a test cartridge for containing the sample and the biosensor, wherein introducing the biosensor into the test cartridge mixes the sample and the biosensor, and wherein the test cartridge further includes:
(i) a reaction chamber for receiving the sample and the biosensor, wherein the reaction chamber has a predetermined internal geometry, and wherein the reaction chamber has been adapted to maximize the detectability of the signal while simultaneously reducing background noise;
(ii) optionally, at least one stabilizer located in the reaction chamber, wherein the stabilizer is operative to minimize shear force damage to the sample and biosensor during mixing of the sample and the biosensor; and
(b) a testing unit adapted to receive the test cartridge, wherein the testing unit further includes a detector for detecting and amplifying the signal generated by the biosensor and wherein detection of the amplified signal is indicative of the presence of the infectious agent; and
(c) wherein detection of the specific infectious agent in the sample occurs in real time.

8) The system of claim 6, wherein the detectable reporter gene is an aequorin reporter gene.

9) The system of claim 6, wherein the biosensor is pre-charged with coelenterazine, and wherein any excess coelenterazine is removed from the biosensor prior to reacting the biosensor with the sample to be tested.

10) The system of claim 6, further comprising means for disrupting individual cells of the infectious agent prior to mixing the sample with the biosensor.

11) The system of claim 10, wherein the means for disrupting individual cells of the infectious agent prior to mixing the sample with the biosensor further includes at least one of an enzyme operative to release O antigens from the cell surface, a sonicator operative to fragment the cells, a French Press operative to fragment the cells, and a chemical treatment operative to release LPS from the cells of the infectious agent.

12) The system of claim 6, wherein the sample to be tested is derived from beef, poultry, fish, or vegetable matter.

13) The system of claim 6, wherein the specific infectious agent is Escherichia coli.

14) The system of claim 6, wherein the detectable signal further includes light.

15) The system of claim 7, wherein the at least one stabilizer further includes Pluronic F68.

16) The system of claim 7, wherein real time is within about one to five minutes from creation of the sample to be tested.

17) A system for rapidly detecting infectious agents in biological samples, comprising:
(a) at least one sample to be tested for at least one specific infectious agent; and
(b) at least one biosensor, wherein the biosensor is operative to detect a specific infectious agent in the sample to be tested; wherein the biosensor emits a detectable signal when it reacts with the specific infectious agent; wherein the biosensor is a hybridoma cell that naturally expresses an endogenous anti-target antigen specific IgM; wherein the hybridoma cell has been converted to a B cell receptor biosensor by introducing a detectable reporter gene into the cell; and wherein the detectable reporter gene is an aequorin reporter gene.

18) The system of claim 17, further comprising:
(a) a test cartridge for containing the sample and the biosensor, wherein introducing the biosensor into the test cartridge mixes the sample and the biosensor, and wherein the test cartridge further includes:
(i) a reaction chamber for receiving the sample and the biosensor, wherein the reaction chamber has a predetermined internal geometry and at least one highly reflective inner surface; and
(ii) optionally, at least one stabilizer located in the reaction chamber, wherein the stabilizer is operative to minimize shear force damage to the sample and biosensor during mixing of the sample and the biosensor; and
(b) a testing unit adapted to receive the test cartridge, wherein the testing unit further includes a detector for detecting and amplifying the signal generated by the biosensor and wherein detection of the amplified signal is indicative of the presence of the infectious agent; and
(c) wherein detection of the specific infectious agent in the sample occurs in real time; and wherein real time is within about one to five minutes from creation of the sample to be tested.

19) The system of claim 17, wherein the biosensor is pre-charged with coelenterazine, and wherein any excess coelenterazine is removed from the biosensor prior to reacting the biosensor with the sample to be tested.

20) The system of claim 17, further comprising means for disrupting individual cells of the infectious agent prior to mixing the sample with the biosensor.

21) The system of claim 20, wherein the means for disrupting individual cells of the infectious agent prior to mixing the sample with the biosensor further includes at least one of an enzyme operative to release O antigens from the cell surface, a sonicator operative to fragment the cells, a French Press operative to fragment the cells, and a chemical treatment operative to release LPS from the cells of the infectious agent.

22) The system of claim 17, wherein the sample to be tested is derived from beef, poultry, fish, or vegetable matter.

23) The system of claim 17, wherein the specific infectious agent is Escherichia coli.

24) The system of claim 17, wherein the detectable signal further includes light.

25) The system of claim 18, wherein the at least one stabilizer further includes Pluronic F68; and wherein the at least one additive further includes a surfactant.
